(19) United States
(12) Patent Application Publication
(10) Pub. No.: US 2006/0161179 A1
(43) Pub. Date: Jul. 20, 2006

(54) Follicular transplantation device and method

(76) Inventor: Robert M. Kachenmeister, Newport Coast, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/316,357

(22) Filed: Dec. 22, 2005

Related U.S. Application Data
(60) Provisional application No. 60/639,120, filed on Dec. 23, 2004.

(51) Int. Cl. A61B 17/50

(52) U.S. Cl. 606/133

(57) ABSTRACT

A hair transplantation device can include a generally tubular guide configured to locate a hair follicle bulb by sliding over a hair shaft down to an upper surface of a hair follicle. The guide can have a blunt distal end configured not to transect the hair follicle bulb. The hair transplantation device also includes an outer tubular member sized and configured to be advanced around the guide. The outer tubular member has a dilator and a harvester. The device can be used in a follicle transplantation method in which the guide locates a hair follicle bulb, and the outer tubular member is slid over the guide. Once the outer tubular member is aligned with the guide, the harvesting portion is advanced, cutting dermal tissue surrounding the hair follicle. The outer tubular member is then withdrawn with the hair follicle, relocated in a recipient area, and the hair follicle implanted.

### RELATED APPLICATION DATA

This application claims priority to U.S. Provisional Application No. 60/639,120 entitled “FOLLICULAR TRANSPLANTATION DEVICE AND METHOD” filed on Dec. 23, 2004, the entire contents of which is hereby incorporated by reference.

### BACKGROUND OF THE INVENTIONS

1. Field of the Inventions

The present inventions relate to hair transplant devices and, in particular, to devices for harvesting hair follicles from animal dermis and devices for implanting the hair follicles into animal dermis.

2. Description of the Related Art

Hair transplant procedures have been carried out for decades. Conventional hair transplant operations are performed with the following process: harvesting of a piece of scalp in the donor area to obtain a transplanting skin (so called “donor scalp”), pulling and suturing the remaining portion of donor area skin after harvest from the donor area, separating each of the hairs including the hair follicle roots from the donor scalp, making incisions into a bald area, and implanting the separated hairs into the insertions. One significant drawback with the traditional hair transplant method is that the removal of donor scalp leaves large linear scars in the donor area. Because of the undesirability of the scars left by the conventional method, many potential donor areas are undesirable as most people would prefer not to have visible scaring across their arms, legs, or other potential donor areas.

There have been various attempts to solve this scarring problem by focusing on removal and transplant of single hairs in a transplant operation. Typically, these attempted solutions have related to manually operated tools to remove hair follicles with a perforation or cutting blade. The use of such tools can alleviate the scarring problem of the conventional hair transplant method, at least to a certain degree. However, such the hair follicle harvesting tools have other drawbacks. Notably, the perforation or cutting tools require an operation with surgical precision in order to extract a hair follicle because they require positioning a relatively small blade about a still smaller visible hair root. Moreover, due in part to the precision required and in part to an offset between the visible hair root on the scalp and the hair follicle bulb under the scalp, these tools frequently transect, injure, or destroy hair follicle bulbs in removal attempts.

### SUMMARY OF THE INVENTIONS

An aspect of at least one of the embodiments disclosed herein includes the realization that the shaft of a hair of an animal can be used as a guide for removing individual hairs for implantation elsewhere. For example, tubular members can be used in a manner that the tubular member is slid over a hair shaft in a manner that the tubular member is guided, by the hair shaft, to the follicle. As such, the follicle itself can be more accurately located and removed for implantation elsewhere.

In one embodiment of the present invention, a hair removal device is disclosed. The hair removal device comprises a guiding inner tubular member with a blunt distal end and an outer tubular member. Both the inner tubular member and the outer tubular member have distal ends configured to expand a pore around the hair follicle. Once the hair follicle is removed, the pore will quickly return to its normal size. By expanding the pore surrounding the hair follicle rather than cutting tissue, the risk of scarring with the hair removal device is significantly reduced over the conventional techniques. This inner tubular member is configured such that it can be slid over a hair in the donor area and into the dermal tissue into which the hair is rooted. The blunt distal end of the inner tubular member can be advanced until the inner tubular member contacts the upper service of the hair follicle bulb. Therefore the inner tubular member is able to locate the hair follicle bulb using the hair shaft itself as a guide. The outer tubular member is configured to be slidably engaged over the inner tubular member over the hair to be removed. The outer tubular member comprises an inner dilating portion and an outer harvesting portion. At the distal end of the outer tubular member, the inner dilating portion has a honed dilating blade tip, while the outer harvesting portion has a honed tip. Once the outer tubular member is slidably advanced over the inner tubular member to a position in the dermal tissue adjacent the upper surface of the hair follicle bulb, the harvesting portion of the outer tubular member can be advanced around the hair follicle bulb. The harvesting portion is configured so that it will dilate the pore surrounding the hair follicle bulb and cut dermal tissue surrounding the hair follicle bulb, thereby leaving the hair follicle bulb free to be extracted. Once the pore surrounding the hair follicle bulb has been dilated and dermal tissue around the hair follicle bulb has been cut, the outer tubular member can then be retracted along with the hair follicle bulb.

One further aspect of the hair removal device of the present invention relates to the retraction of the outer tubular member with the hair follicle bulb. In this further aspect, a suction device is used to withdraw the hair follicle bulb. The suction device can be a generally cylindrical segment with a manually operable vacuum creating membrane as described in detail herein, or other types of suction or extraction devices can be used.

The hair removal device, as described above, can also be used as a hair transplant device in another embodiment of the present invention. When used as a transplant device, once the hair follicle is removed, as described above, the follicle is transported to a recipient site or bald area that has been prepared to accept a hair follicle according to known techniques. The hair follicle is then implanted into the recipient area through insertion of the hair transplant device into the dermal tissue of the recipient area and extrusion of the hair follicle from the hair transplant device. To assist with the extrusion of a hair follicle, the hair transplant device can further comprise a plunger configured to fit in an inner diameter of the outer tubular member and extrude the hair follicle once the hair transplant device has been positioned in the dermal tissue of the recipient area.

Another embodiment of the present invention is a hair removal method using a hair removal device as described above. The hair removal method generally comprises the steps of sliding an inner tubular member over a hair in a donor area. As noted above, the inner tubular member can be advanced until it reaches an upper surface of the hair follicle bulb. Preferably, the inner tubular member will be configured to ease this positioning by having an inner diameter that allows the inner tubular member to fit around the hair shaft but is not large enough to allow it to fit around the hair follicle. This configuration allows the inner tubular member to use the hair shaft as a guide to locate the hair follicle bulb under the visible surface layer of skin. Once the inner tubular member reaches the upper surface of the hair follicle, the method further comprises sliding an outer tubular member over the inner tubular member. The outer tubular member can be configured with an inner dilating portion and an outer harvesting portion configured such that the outer harvesting portion can be slidably advanced or retracted relative to the inner dilating portion. This configuration allows the outer tubular member to dilate the dermal tissue surrounding the hair and hair follicle using a dilating distal end of the inner dilating portion, then dilate the pore surrounding the hair follicle bulb and cut the dermal tissue surrounding the hair follicle by further advancing the outer harvesting portion around the follicle. Finally, the method comprises withdrawing the outer tubular member with a hair follicle. In another aspect of the hair removal method, the step of withdrawing the outer tubular member with a hair follicle can be accomplished with the aid of a suction device.

An additional embodiment of the present invention is a method for hair transplantation using a hair transplant device. The method of hair transplant generally follows the method of hair removal discussed above for removal of a hair follicle from a donor area, followed by the additional steps of transporting the removed hair follicle to a recipient location, implanting the hair follicle from the new recipient location, and performing post-operative treatment to the both donor and recipient areas as necessary. The hair transplantation device can be used to transport and implant the removed hair follicle in the recipient area. The hair can be implanted in the recipient area by inserting the hair transplant device into the dermal tissue according to known implantation techniques and extruding the hair follicle bulb. A plunger can be used to extrude the hair follicle bulb from the hair transplant device.

### BRIEF DESCRIPTION OF THE DRAWINGS

FIG. 1 is an exploded view of a hair transplantation device in accordance with an embodiment, and including an inner tubular member and an outer tubular member;

FIG. 2A is a perspective view of the inner tubular member of FIG. 1;

FIG. 2B is a perspective view of the outer tubular member of FIG. 1;

FIG. 2C is a perspective view of a donor site preparation instrument that can be used with the hair transplantation device of FIG. 1;

FIG. 2D is a depiction of donor and recipient areas of a patient for hair transplant;

FIG. 3A is a side view of the donor site preparation instrument illustrating the optional procedure of applying the instrument to a hair in a donor area;

FIG. 3B is a side view of the inner tubular member of FIG. 1 applied to a hair in a donor area;

FIG. 4A is a perspective view of the hair transplantation device with the outer tubular member slidably advanced over the inner tubular member;

FIG. 4B is a cross-sectional view of the hair transplantation device taken along line 4B-4B of FIG. 4A;

FIG. 4C is a perspective view of the hair transplantation device with the harvesting portion of the outer tubular member advanced over a hair follicle bulb;

FIG. 4D is a cross-sectional view of the hair transplantation device taken along line 4D-4D of FIG. 4C;

FIG. 4E is a perspective view of a hair donor area after hair removal by the hair transplantation device of FIGS. 1-4;

FIG. 5A is a perspective view of a modification of the hair transplantation device of FIGS. 1-4 with an outer tubular member having a harvesting portion with a modified collar advanced over a hair follicle bulb;

FIG. 5B is a cross-sectional view of the hair transplantation device taken along line 5B-5B of FIG. 5A;

FIG. 5C is a perspective view of the outer tubular member of FIG. 5A with the inner tubular member and the inner portion of the outer tubular member removed;

FIG. 6A is a perspective view of a suction device that can be used with the transplantation device of FIG. 5;

FIG. 6B is a perspective view of the suction device of FIG. 6A in sealing connection with the outer tubular member of FIG. 5;

FIG. 6C is a perspective view of the suction device of FIG. 6A in sealing connection with the outer tubular member of FIG. 5 as applied to a hair in a donor area;

FIG. 6D is a perspective view of the suction device of FIG. 6A in sealing connection with the outer tubular member of FIG. 5 after removal of the hair from the donor area;

FIG. 7 is a flow chart illustrating steps of a method for hair removal that can be performed with the devices of FIGS. 1-6 or other devices;

FIG. 8 is a perspective view of a hair recipient area before implantation of a hair;

FIG. 9 is a side view of a hair recipient area with an incision cut in preparation for a hair transplant;

FIG. 10A is a side view of a hair recipient area with the outer tubular member of FIGS. 1-4 positioned to transplant a hair;

FIG. 10B is a cross-sectional view of the outer tubular member of FIGS. 1-4 and a hair extrusion plunger for expelling a hair from the outer tubular member;

FIG. 10C is a side view of the hair extrusion plunger of FIG. 10B;

FIG. 11 is a side view of a hair recipient area after receipt of a transplanted hair follicle;

FIG. 12A is a flow chart illustrating steps of a method of transplanting a hair follicle that can be performed with the devices of FIGS. 1-6 or other devices;

FIG. 12B is a flow chart illustrating steps of a method of implanting a hair follicle into a recipient area using an outer tubular member of FIGS. 1-6.

### DETAILED DESCRIPTION OF THE PREFERRED EMBODIMENT

Turning now to the drawings provided herein, a more detailed description of the embodiments of the present inventions is provided below. It should be noted, however, that while some embodiments have all of the advantages identified herein, other embodiments may only realize some but not all of the advantages.

Hair Transplantation Device

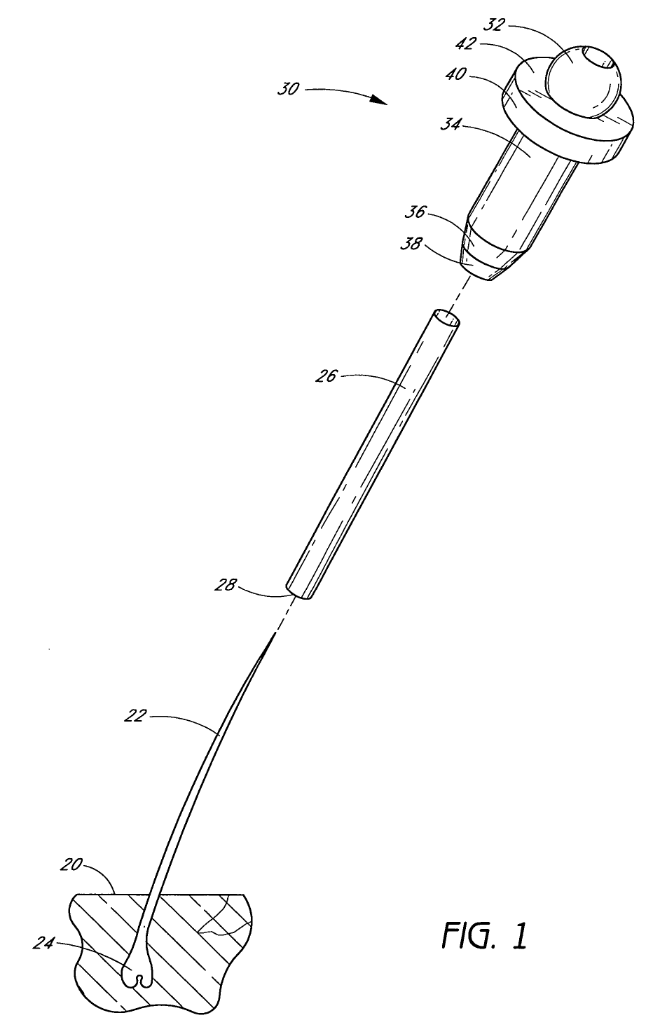

With reference to FIGS. 1-6, various embodiments of a hair transplantation device are depicted. FIG. 1 depicts an exploded perspective view of a hair transplantation device. The hair transplantation device comprises an inner tubular member 26 and an outer tubular member 30.

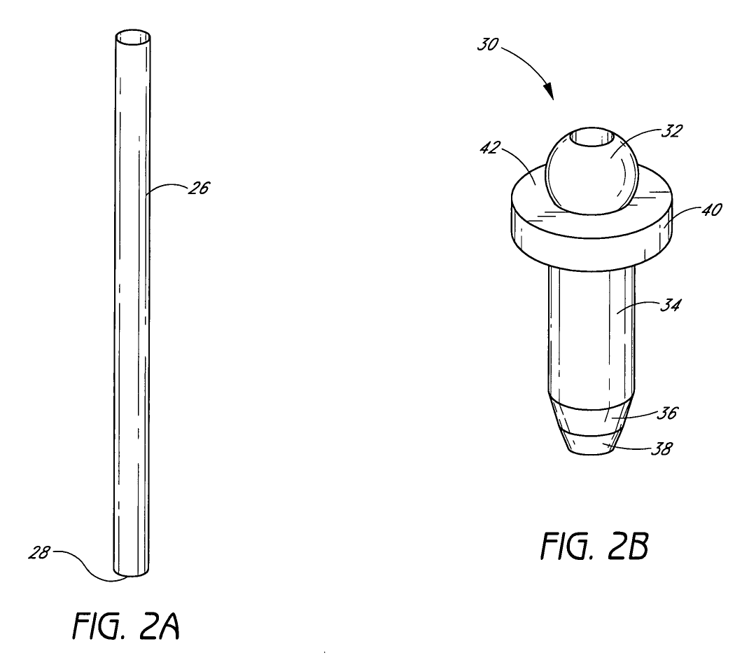

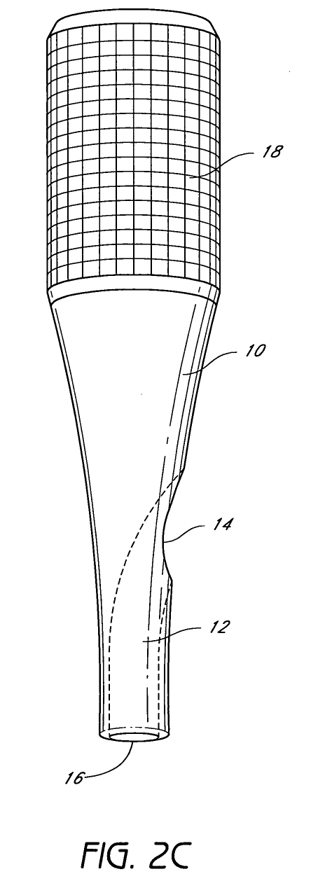

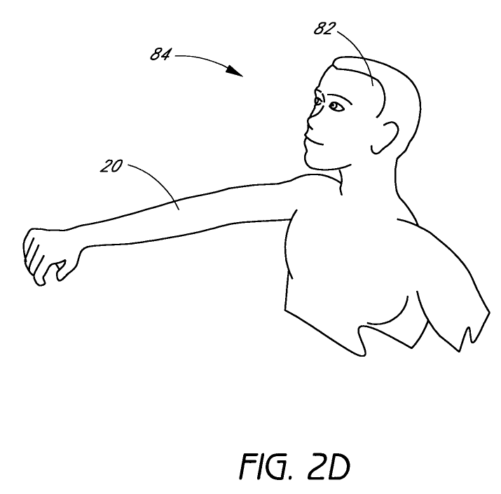

The inner tubular member 26 of the hair transplantation device of FIG. 1 is depicted in FIG. 2A. The inner tubular member 26 can have a generally tubular shape allowing it to be slidably advanced over a shaft of hair to provide a guide to the hair follicle bulb. In some embodiments, the inner diameter can be about the same or slightly larger than an average diameter of animal hair. For example, in some embodiments, the inner diameter of the inner tubular member 26 can be about 0.001″ to 0.003″ larger than the diameter of a human hair. However, other sizes can also be used. The wall thickness of the inner tubular member 26 can be any size. In some exemplary embodiment, the wall thickness of the inner tubular member 26 can be about the diameter of an average human hair. However, other wall thicknesses can also be used.

One advantage provided by this generally tubular shape is that it facilitates location of a hair follicle under the surface of the skin. The inner tubular member 26 can have a blunt distal end 28. The blunt distal end 28 provides a further advantage that when the inner tubular member 26 is advanced over a hair shaft to a hair follicle bulb 24, the blunt distal end 28 of the inner tubular member 26 will resist cutting the hair follicle bulb. The terms inner tubular member 26 and guide 26 are used interchangeably herein to describe the inner tubular member 26. In such a mode of operation, the shaft of the hair 22 itself provides a guide for the placement of the inner tubular member 26 over the hair 22, and can guide the inner tubular member accurately toward the follicle bulb 24.

The inner tubular member 26 can be configured such that an inner diameter is sized to fit around a typical hair 22 shaft but not around a hair follicle bulb 24. An inner diameter sized according to this configuration will prevent the inner tubular member 26 from being advanced past an upper surface of the hair follicle bulb 24 of the hair 22 shaft. Advantageously, this diametric configuration aids in the location of a hair follicle bulb as the guide 26 is slid down a hair 22 shaft. Further, this configuration advantageously reduces the risk of injury to the hair follicle bulb as the inner tubular member 26 can not be advanced far enough to transect the hair follicle bulb 24.

The inner tubular member 26 can have an outer diameter configured to pass through dermal tissue and dilate a pore surrounding a hair follicle with minimal tearing or removal of dermal tissue. One advantage realized by this outer diameter configuration is that post-operative scarring due to hair removal with the device of the present invention is reduced. Advantageously, as a result, previously undesirable hair donor areas such as a patient's arms and legs can more easily be used as hair donor areas.

In some embodiments, the inner diameter of the guide 26 is less than about 0.5 mm and the outer diameter of the guide is about 0.5 mm. Such a diametric sizing of the inner tubular member 26 allows its application to human patients having hair 22 of average diameters. However, it should be noted that there is relatively little variation in hair 22 shaft and follicle bulb 24 size among people generally. While the hair transplantation device is discussed generally herein in the context of its application to a human patient, other embodiments of the device may include diametric sizings of the guide 26 to facilitate application of the hair transplantation device to animals.

The outer tubular member is depicted in FIG. 2B. The outer tubular member 30 can comprise an inner dilating portion 32 and an outer harvesting portion 34. The inner dilating portion 32 and the outer harvesting portion 34 are interchangeably referred to as the dilator 32 and the harvester 34, respectively, herein. As depicted, the outer tubular member 30 is formed of the dilating portion 32 and the harvesting portion 34 joined as a two-piece assembly or a single piece having the same dimensions, but it is recognized that the outer tubular member 30 can alternately comprise a dilator 32 and a harvester 34 as two separable concentric generally tubular segments.

The outer harvesting portion 34 can be slidably advanced and retracted with respect to the dilator 32. The inner dilating portion 32 of the outer tubular member 30 may have a honed distal end 38. One advantage of the honed distal end 38 is that it allows the outer tubular member 30 to dilate the dermal tissue surrounding a hair follicle bulb. Specifically, the outer tubular member 30 may dilate the pore surrounding a hair follicle. The dilation of dermal tissue provided by the outer tubular member 30 allows the hair removal device of the present invention to remove hair follicles with a substantially reduced risk of scarring as compared with a cutting or perforation tool.

Preferably, the diameters of the inner tubular member 26, the dilator 32, and the harvester 34 of the outer tubular member 30 are closely matched to allow them to be slidable with respect to one another such that the outer tubular member 30 may be advanced over the inner tubular member 26 into the dermis, and the harvester 34 further advanced over the dilator 32. For example, the inner dilating portion 32 of the outer tubular member 30 may have an inner diameter sized and configured to allow the outer tubular member 30 to be slidably advanced over the inner tubular member 26.

The inner diameter of the dilator 32 can be closely matched to the outer diameter of the inner tubular member 26 such that a small clearance exists between the inner tubular member 26 and the outer tubular member 30. Likewise, the dilator 32 preferably has an outer diameter closely matched to an inner diameter of the harvester 34. Advantageously, this close matching of diameters allows the inner tubular member 26 to serve as a guide for the outer tubular member 30 to a hair follicle bulb so that when slidably advanced over the inner tubular member 26, the outer tubular member 30 is substantially centered with respect to the hair follicle bulb. Preferably, the inner tubular member 26 comprises a 27 gauge flexible tube, the dilating portion 32 of the outer tubular member 30 comprises a 22 gauge tube, and the harvesting portion of the outer tubular member 30 comprises an 18 gauge tubular member.

The outer harvesting portion 34 of the outer tubular member 30 can be a substantially tubular segment that is slidably engaged over the inner dilating portion 32 of the outer tubular member 30. The length of the slidable travel of the harvester 34 with respect to the inner dilating portion 32 of the outer tubular member may be restricted by a feature such as a flange or stop on the inner dilating portion 32. Optionally, the length of recommended slidable travel can be indicated on the sides of tubular members 26, 30 with indicia (not shown) or with any other technique. A flange, stop, or indicia can be configured to allow the outer harvesting portion 34 to slidably advance only as far as necessary (or in the case of indicia, provide a visual indicator indicating a sufficient depth) to dilate the pore surrounding the hair follicle bulb and cut the dermal tissue immediately surrounding the hair follicle. In such a configuration, the flange, stop, or indicia would allow the harvester 34 to slidably travel a distance corresponding to the average length between an upper surface of a hair follicle bulb and a lower surface of a hair follicle bulb. One advantage provided by such a restriction in slidable travel is that it allows the hair follicle to be removed from the adjacent dermal tissue with minimal cutting of the tissue, thereby reducing the risk of scarring and promoting post-operative healing.

The outer harvesting portion 34 of the outer tubular member 30 can have a sharpened honed blade at its distal end 36 allowing it to dilate the pore immediately surrounding the hair follicle bulb 24 and cut dermal tissue immediately surrounding the hair follicle bulb 24. An inner diameter of the distal end 36 of the harvester 34 is preferably sized to be slightly larger than an average diameter of a hair follicle bulb 24, although, as noted above, in alternate embodiments, the harvester 34 can be sized and configured to harvest hair follicles of other-than average size, or animal hair follicles. An inner diameter of the distal end 36 of the harvester 34 can be approximately 1 mm, however, other dimensions can also be used.

This honed blade allows the outer harvesting portion 34 of the outer tubular member 30 to separate a hair follicle bulb from the surrounding dermal tissue. This configuration advantageously allows the harvester of the outer tubular member 30 to be advanced around the hair follicle bulb thereby separating the hair follicle bulb from the surrounding dermal tissue.

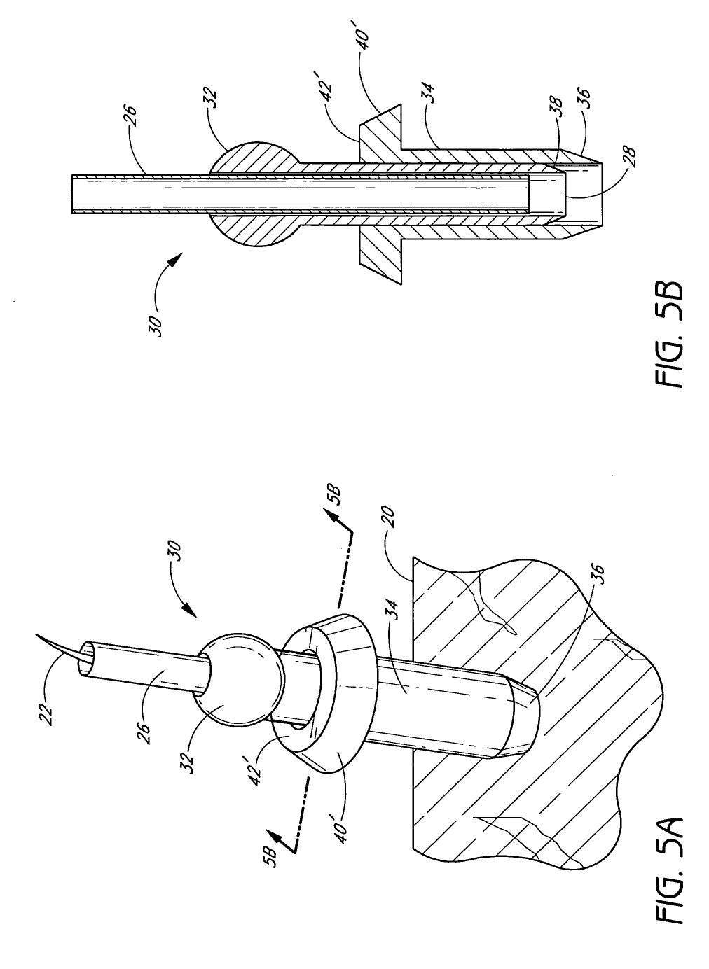

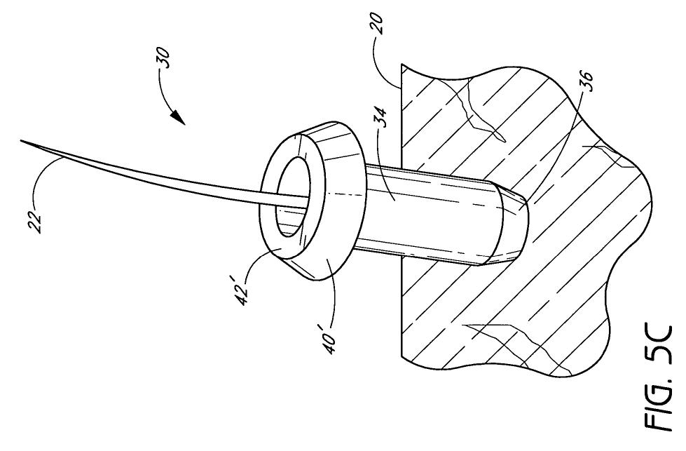

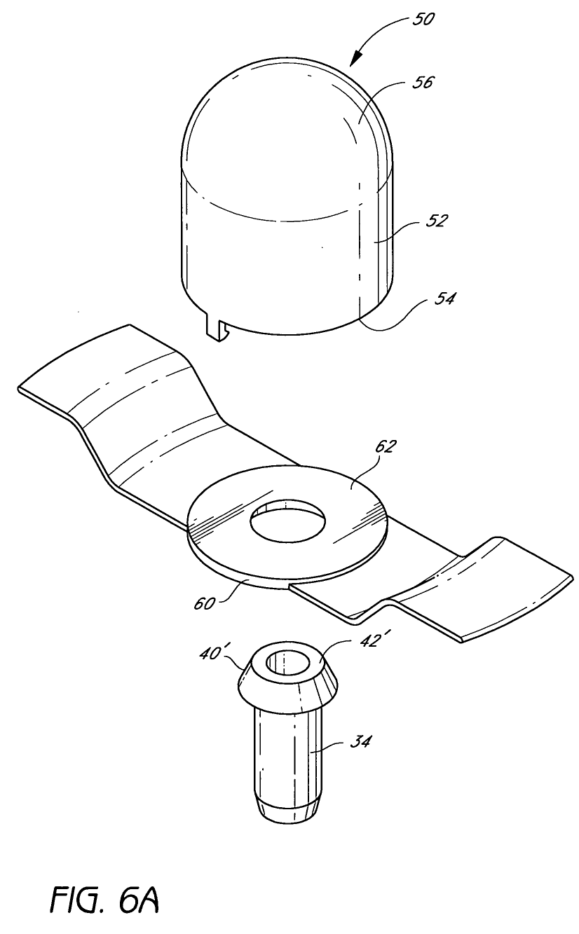

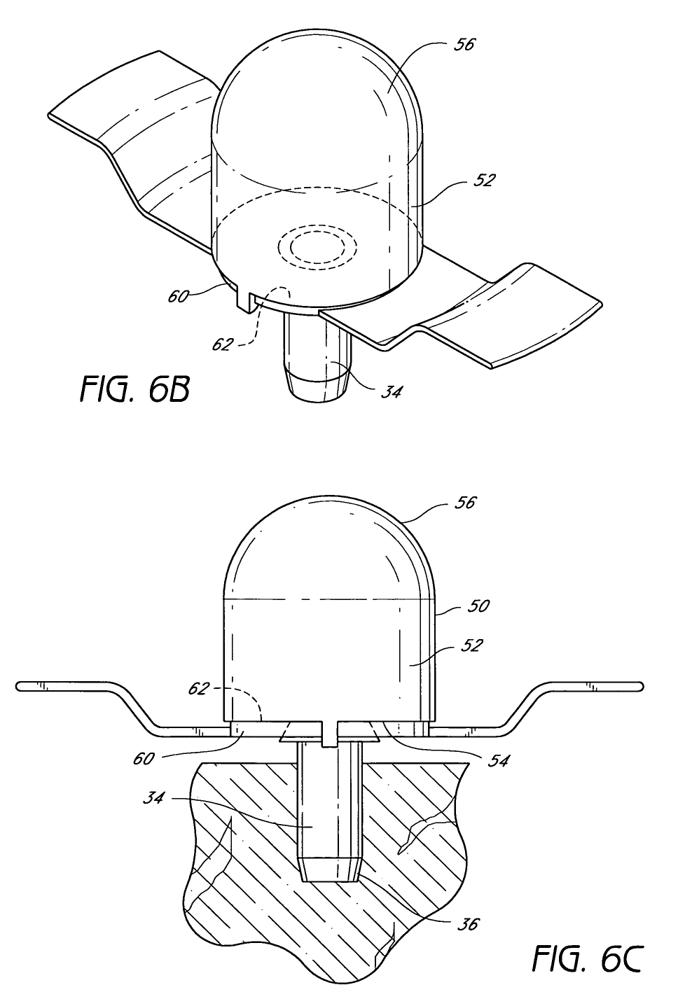

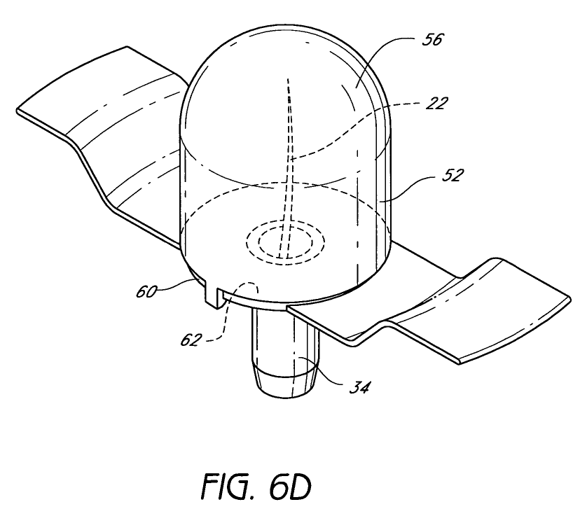

The outer harvesting portion 34 of the outer tubular member 30 can have an enlarged collar 40 at its proximal end. In some embodiments, as depicted in FIGS. 1, 2, and 4, the collar 40 has a substantially disc-shaped profile. In other embodiments, as depicted in FIGS. 5 and 6, the collar 40 can be shaped to mate with a suction device as discussed with reference to FIG. 6. The collar 40 can have a sealing surface 42 for mating with a suction device in certain embodiments of the present invention.

With reference to FIG. 2C, an optional donor site preparation instrument 10 is depicted. The instrument 10 can be used in conjunction with the hair transplantation device of FIG. 1, or other hair transplantation devices. The instrument 10 includes an elongated tool body having a lower opening 16, an upper opening 14, and a passage 12 connecting the openings 16, 14. The instrument 10 has a handle 18 to allow a medical practitioner to easily grasp and manipulate the tool. The tool body is preferably rigid such that it can penetrate the dermal tissue of a person without crumpling.

With reference also to FIG. 2D, the present hair transplantation device can be used to transplant hair from a donor area 20 to a recipient area 82. While the donor area 20 is depicted as the arm of a hair transplant patient 84, it is contemplated that other donor areas 20 could be considered. One advantage of the systems and methods of the present inventions is hair transplantation with a significantly reduced risk of visible scarring. Therefore, relatively visible skin areas such as arms and legs can be considered when a patient 84 and a medical practitioner are selecting donor areas 20.

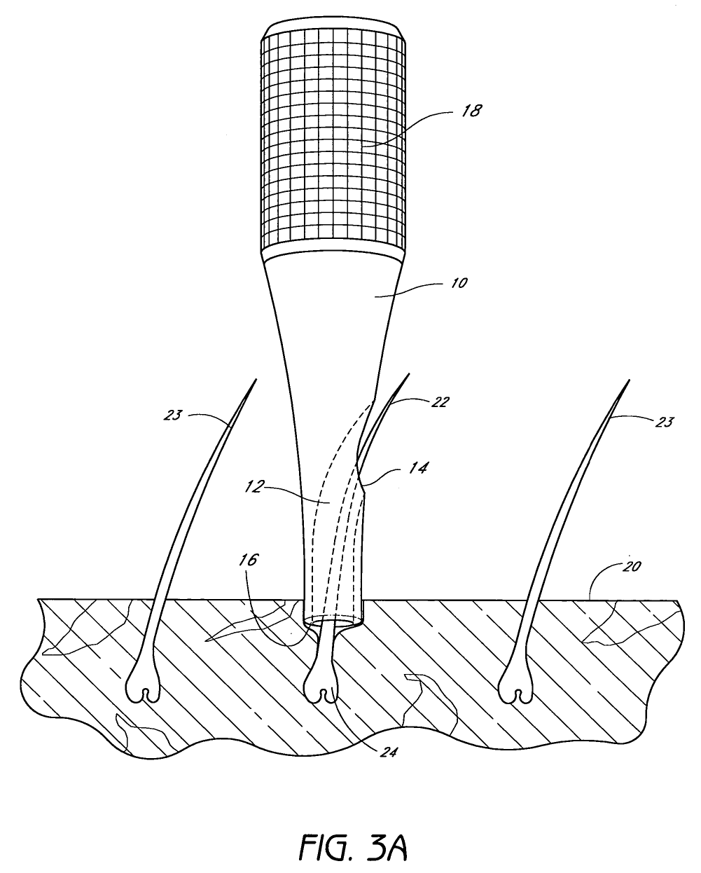

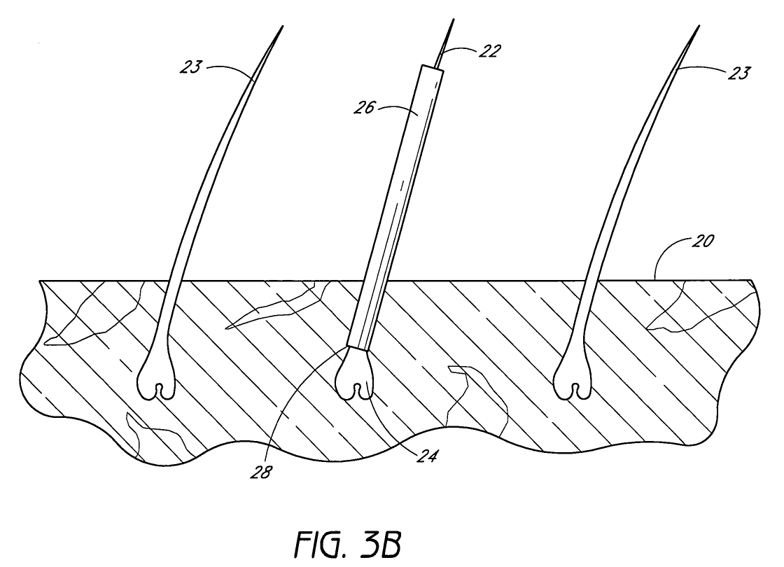

With reference to FIGS. 2C and 3A an optional donor site preparation instrument is illustrated as preparing a hair 22 to be removed with a hair transplantation device. The instrument 10 can be positioned over the hair 22, and advanced toward the skin such that the hair 22 is inserted through the lower opening 16.

In some embodiments, the instrument 10 is advanced such that the hair 22 advances through the passage 12 and out the upper opening 14. Once the hair 22 exits the upper opening, it can be gripped with forceps or another gripping tool to facilitate advancement of the preparation instrument 10 down the hair 22.

The lower opening 16 of the instrument is advanced through the skin and down along the hair 22 shaft. The end of the instrument 10 adjacent the lower opening 16 preferably has a polished surface finish, and can include a radiused edge such that it spreads or dilates the dermal tissue immediately surrounding the hair 22 without cutting or tearing dermal tissue.

The lower opening 16 can be sized and configured to dilate a pore surrounding a hair 22 to prepare that pore for receiving a hair transplantation device. The instrument need not be advanced into the dermal tissue completely to a depth of the hair follicle bulb 24, but can be advanced partway as depicted. Advantageously, the preparation instrument 10 spreads the dermal tissue surrounding a donor hair 22, thus facilitating application of the hair transplantation device as discussed with reference to FIGS. 3B, 4, and 5.

FIG. 3B depicts an inner tubular member 26 of the hair removal device as used to locate a hair follicle bulb 24 below the surface of the skin at a hair donor area 20. As depicted, the inner tubular member 26 is slid over the hair 22 and advanced into the dermal tissue.

Before application of the inner tubular member 26 to the hair 22, the subcutaneous dermal tissue can be injected with an anesthetic injection to promote tissue turgidity and ease passage of the guide 26 through the dermal tissue. In some embodiments, a sufficient amount of anesthetic is injected so as to palpably harden the surrounding tissue. This provides the further advantage of stiffening the tissue which eases the harvesting procedure.

Additionally, before application of the inner tubular member 26, the subcutaneous dermal tissue can be spread using a preparation instrument 10 as illustrated in FIG. 3A. In some embodiments including the use of anesthetic, preparation instrument 10 can be used the before or after the injection of the anesthetic. These preparatory techniques can ease application of the inner tubular member 26 especially where a patient has relatively tough dermal tissues which would tend to cause the inner tubular member 26 to bow or buckle.

The inner tubular member 26 can have a blunt distal end 28. The blunt distal end 28 of the inner tubular member 26 can have an inner diameter sized such that it can slide over the outer surface of the hair shaft, but not over the hair follicle bulb 24. Therefore the inner tubular member 26 is stopped at an upper surface of the hair follicle bulb. The donor area 20 of skin also features adjacent hairs 23. Once the hair 22 in the donor area 20 has been removed, the adjacent hairs 23 of the donor area 20 can also be removed in the same or similar manner.

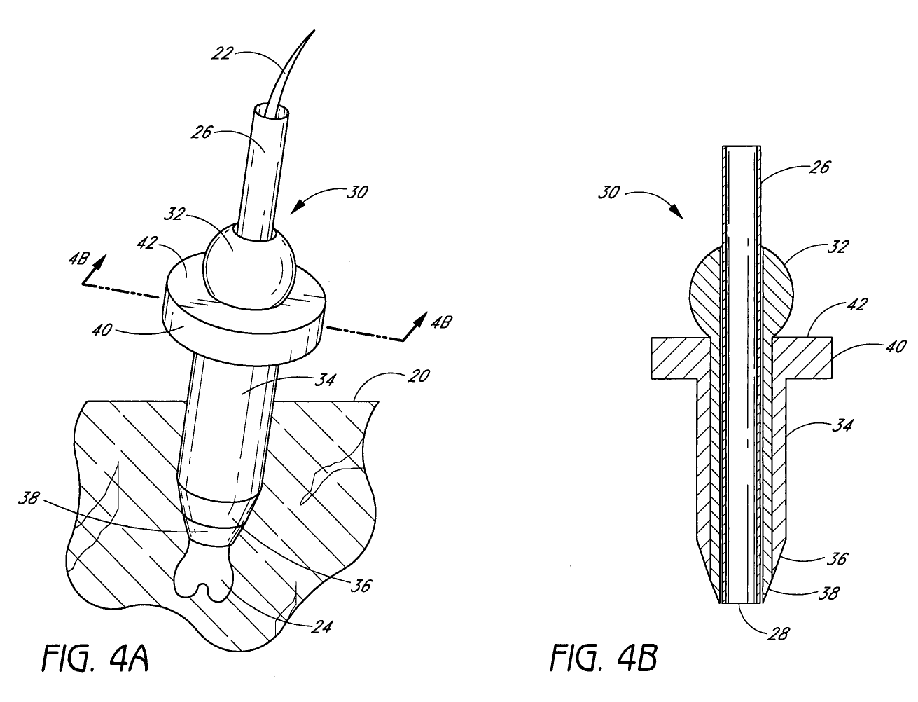

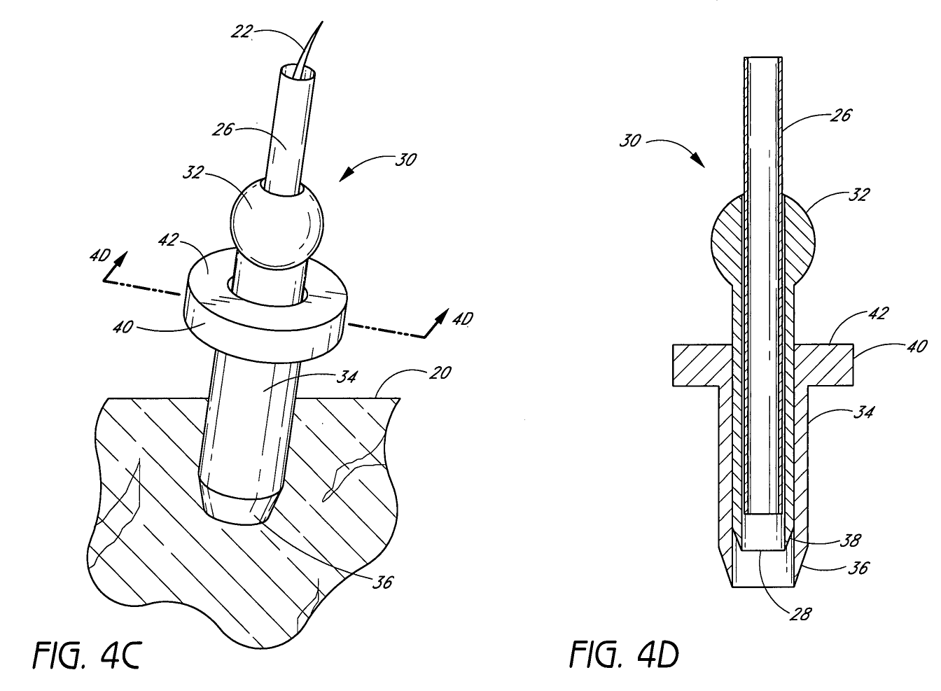

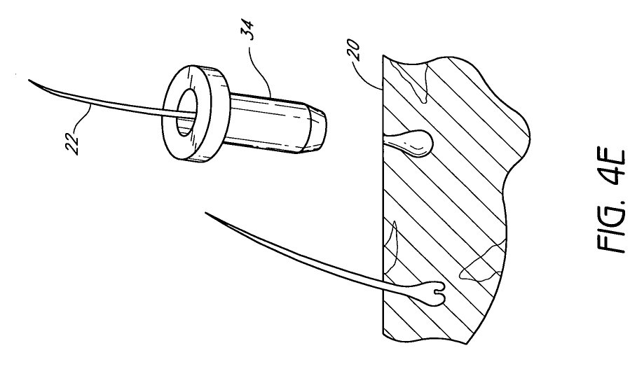

FIG. 4A depicts an outer tubular member 30 slid over an inner tubular member 26 over a hair 22 in a donor area 20 of skin. As depicted, the outer tubular member 30 has been advanced into the dermis of the donor area until its distal end has reached an upper surface of a hair follicle bulb 24. As shown in FIG. 4, the distal end 38 of the inner dilating portion 32 of the outer tubular member 30 has separated the dermal tissue surrounding the hair 22 down to the hair follicle 24. The distal end 38 has dilated a pore surrounding the hair 22. One advantage of such dilation is that it reduces unnecessary cutting or tearing of the dermis which further reduces scarring when the hair follicle bulb 24 is removed.

The present hair removal device can feature various indicators to show that the outer tubular member 30 has been advanced to the upper surface of the hair follicle. For example, the inner tubular member 26 can have a marking or indicia on its proximal end that indicates when the outer tubular member 30 is advanced such that the distal end of the outer tubular member has reached the distal end of the inner tubular member 38 at an upper surface of a hair follicle bulb 24. Alternately, advancing the outer tubular member 30 to the appropriate depth can result in a tactile cue (such as increased resistance as corresponding features on the inner tubular member and the outer tubular member mate) or another tactile or audible cue (a “click” sound or vibration indicating that the distal ends 28, 38 of the inner tubular member 26 and the outer tubular member 30 have aligned).

In the configuration depicted in FIGS. 4A and 4B, the inner tubular member 26 has guided the outer tubular member 30 into a centered alignment with the hair follicle bulb 24. One advantage of the illustrated embodiments of hair transplantation device is that this centered alignment of the outer tubular member 30 with the hair follicle bulb 24 is created even where the hair follicle bulb 24 is offset from the location where the hair 22 visibly exits the donor area 20 skin. Thus, this centered alignment reduces the risk that a hair follicle bulb 24 will be damaged by the hair transplantation device.

FIG. 4B is a cross-sectional view of the inner tubular member 26 and the outer tubular member 30 engaged in the position depicted in FIG. 4A. As is depicted in FIG. 4B, in this cross-sectional view, it is apparent that the outer diameter of the inner tubular member 26 is configured such that it is closely matched with the inner diameter of the inner dilating portion 32 of the outer tubular member 30. Also, as depicted, the inner dilating portion 32 of the outer tubular member 30 is slidably engaged with the outer harvesting portion 34 of the outer tubular member 30. Thus, the outer harvesting portion 34 can be slidably advanced into the dermal tissue surrounding the hair follicle bulb 24 after the inner dilating portion 32 has followed the inner tubular member 26 as a guide to the upper surface of the hair follicle bulb 24.

FIG. 4C depicts a sliding advancement of the outer harvesting portion 34 of the outer tubular member 30 such that the distal end 36 of the outer harvesting portion 34 dilates the pore and cuts the dermal tissue surrounding the hair follicle bulb of the hair 22. As noted above, the outer tubular member 30 can have a sliding restriction such as a latch or other stop, or a marking or indicia to limit or to indicate a recommended limit of the sliding travel of the outer harvesting portion 34 relative to the inner dilating portion 32. One advantage of such a feature is that the outer harvesting portion 34 of the outer tubular member 30 can cut only the dermal tissue required to free the hair follicle from the dermis. This limited cutting of dermal tissue reduces potential for scarring and expedites post-operative healing. The positioning of the limit or marking or indicia can be determined through routine experimentation directed to the normal, average, or maximum sizes of animal or human hair follicles.

FIG. 4D is a cross-sectional view of the inner tubular member 26 and the outer tubular member 30 of the hair removal device in the position shown in FIG. 4C. FIG. 4D depicts the outer harvesting portion 34 of the outer tubular member 30 slidably advanced with respect to the inner dilating portion 32. Proper alignment with the hair follicle bulb 24 has been maintained by the guiding inner tubular member 26 and the inner dilating portion 32 of the outer tubular member 30 such that the distal end 36 of the outer harvesting portion 34 cuts only surrounding dermal tissue rather than transecting the hair follicle bulb during sliding advancement as depicted in the configuration depicted in FIGS. 4C and 4D.

Once the outer harvesting portion 34 of the outer tubular member 30 has been advanced, thereby cutting the dermal tissue around the hair follicle bulb 24, the inner tubular member 26 and the dilating portion 32 of the outer tubular member can be withdrawn.

For example, FIG. 4E shows a view of the donor area 20 after a hair 22 has been removed with a hair transplantation device. The inner tubular member 20 and the inner dilating portion 32 of the outer tubular member 30 have been withdrawn from the outer harvesting portion 34.

In embodiments of hair transplantation device including a sliding restriction between the dilator 32 and the harvester 34 of the outer tubular member 30, the restriction can be configured to allow withdrawal of the dilator. For example, in various embodiments, it is contemplated that rotation of the dilator 32 to a certain orientation relative to the harvester 34 can allow its separation and withdrawal from the harvester, or the sliding restriction can be configured to break away upon application of a withdrawal force on the dilator 32, allowing the dilator 32 to be withdrawn. This removal of the inner components exposes a larger surface of the hair 22 for a medical practitioner to grasp during the removal process.

With continued reference to FIG. 4E, the outer harvesting portion 34 of the outer tubular member has been withdrawn from the skin in the donor area 20. A practitioner can gently pull the outer tubular member 34 away from the skin in the donor area 20 while grasping the hair 22 manually, with forceps, or with some other instrument. To aid withdrawal, the practitioner can gently rotate the outer harvesting portion 34 while pulling.

FIGS. 5A and 5B depict a sliding advancement of an outer harvesting portion 34 of the outer tubular member 30 having a modification to the collar, the modified collar being identified generally with the reference numeral 40′. The sliding of the outer harvesting portion 34 can be substantially as described with respect to FIGS. 4C and 4D. But, in FIGS. 5A and 5B, the collar 40′ of the harvester 34 includes a tapered edge such that the collar 40′ is generally tapered or generally trapezoidal in cross section, as depicted in FIG. 5B. However, other configurations can also be used. The modified collar 40′ can have a sealing surface 42′ that can be configured to sealingly couple to a suction device, or other devices.

FIG. 5C depicts the hair transplantation device having an outer harvesting portion 34 with a modified collar 40′ once the inner tubular member 26 and the dilating portion 32 have been withdrawn. Once the outer harvesting portion 34 of the outer tubular member 30 has been advanced, thereby cutting the dermal tissue around the hair follicle bulb 24, the inner tubular member 26 and the dilating portion 32 of the outer tubular member can be withdrawn and the outer harvesting portion 34 removed from the donor area 20. This withdrawal is substantially as described with respect to FIG. 4E. However, the removal of the outer harvesting portion 34 and hair 22 from the donor area 20 can either be manually performed as described with respect to FIG. 4E, or it can be assisted by the use of a suction device, as described with respect to FIG. 6.

In some embodiments, a suction device can be used in conjunction with the hair transplantation device to remove the hair follicle bulb 24. FIG. 6A depicts an exemplary suction device 50 configured for use with the outer tubular member 30.

The suction device 50 can comprise a housing 52, a sealing edge 54, and an elastic domed surface 56. However, other configurations can also be used. As depicted, the suction device 50, features a substantially cylindrical housing 52 with a lower end defining a sealing edge 54.

An upper end of the housing 52 can include a depressible or deformable elastic domed surface 56. In operation, the elastic domed surface 56 of the suction device 50 can be depressed to a lower position, thereby reducing the interior volume of the suction device 50 as compared to the raised position depicted in FIG. 6A. The elastic domed surface 56 is biased to return the raised position.

The aperture in the sealing surface 62 has a tapered profile configured to couple with the modified collar 40′ of the harvester 34. While mating tapered profiles are illustrated for the mount plate 60 and the collar 40′, in other embodiments, other configurations of mating profiles could be used. Advantageously, the matched profiles of the collar 40′ and the mount plate 60 aperture facilitate a rapid sealed connection between the harvester 34 and the suction device 50.

As depicted in FIG. 6A, the suction device can be used in conjunction with a mount plate 60 to interface with the harvester 34. The mount plate 60 includes a sealing surface 62 configured to mate with the sealing edge 54 of the suction device. The mount plate 60 can also include an aperture through the sealing surface 62 configured to receive the collar 40′ of the harvester 34. With the elastic domed surface 56 depressed into a lower position, the suction device 50 can be applied to the mount plate 60 as shown in FIG. 6B such that the sealing edge 54 of the suction device 50 is sealingly connected with the sealing surface 62 on the mount plate 60.

The mount plate 60 can be positioned on the harvester 34 such that the aperture in the mount plate 60 is coupled with the collar 40 of the harvester 34 and the sealing surface 42 of the collar 40′ is positioned in the suction device 50. This positioning establishes a sealing connection between the suction device 50 and the harvester 34. The elastic domed surface 56 of the suction device 50 can be released. The bias in the elastic domed surface 56 returns the elastic domed surface to its raised configuration as depicted in FIGS. 6A and 6B, and the resulting enlargement of the interior volume of the suction device 50 creates a vacuum in a cavity defined by the inner diameter of the outer tubular member 30, thus aiding in the extracting of the hair with the follicle from the dermal tissue.

If sufficient suction is created by the suction device 50 to pull the hair follicle bulb all the way through the harvester 34, the hair follicle bulb will be contained within the suction device 50. The hair follicle can easily be recovered from the suction device 50 unharmed.

Alternately, the hair transplantation device can also include an internal shield such as a mesh screen having mesh spacing smaller than an average diameter of a hair follicle bulb to prevent the hair follicle bulb from being extracted all the way through the outer tubular member 30 when its removal is assisted with a suction device 50.

Once the hair and its follicle bulb have been separated from the dermal tissue in the donor area, the harvester 34 can be gently removed from the donor area. FIG. 6D illustrates the harvester 34, suction device 50, and hair 22 removed from the donor area 20. A gentle twisting motion can assist removal of the harvester 34 from the donor area.

Hair Removal Method

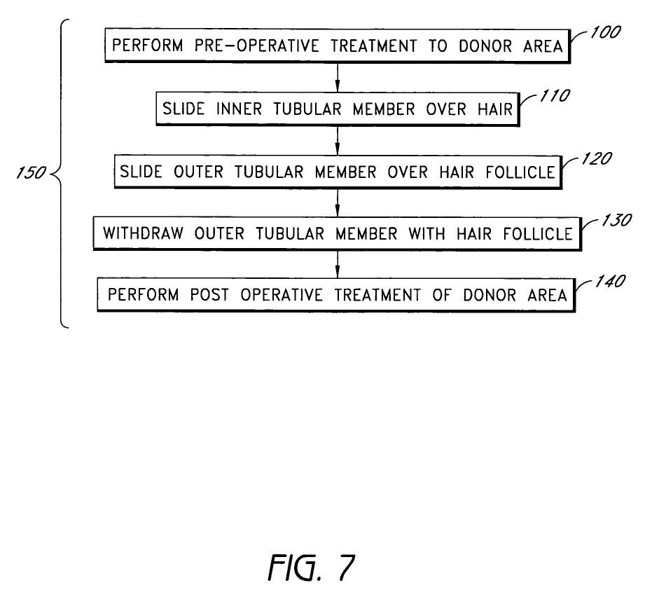

With reference to FIG. 7, an exemplary method for removing hair is described below. The steps of this exemplary method are illustrated in flow chart format in FIG. 7.

The method 150 of removing a hair follicle 150 can comprise the steps of performing pre-operative treatment to a donor area, sliding an inner tubular member over a hair, sliding an outer tubular member over the hair follicle, withdrawing the outer tubular member with the hair follicle, and performing post-operative treatment of the donor area 140. However, other arrangements of these steps, and other steps can also be used in combination with or in lieu of these steps.

In step 100, in which pre-operative treatment of a donor area can be performed, an area of a patient's body is chosen as a donor area, the skin is prepared with an antiseptic solution, and the subcutaneous tissues can optionally be anesthetized with a local anesthetic. The local anesthetic can be, for example, a dilute mixture of lidocaine and saline, although other anesthetics can also be used. In some embodiments, the subcutaneous tissues are over inflated by the anesthetic injection to promote tissue turgidity and stability of the tissue during harvest.

Preferably the chosen donor area has a relatively high hair concentration so that a single anesthetic injection can be applied for removal of multiple hairs through repeating the steps of the method. Advantageously, since the present hair removal methods can result in substantially no scarring of the donor area, the donor area can be chosen from routinely exposed areas such as skin on a patient's arms, legs, or back.

The risk of unsightly scarring caused by several other hair removal and transplant methods had previously rendered these areas essentially unusable as hair donor areas. One further advantage of the tissue turgidity provided by the anesthetic injection is that it reduces the risk of scarring with the hair removal method. The tissue turgidity created by the pre-operative treatment of step 100 facilitates sliding the inner tubular member over the hair and through the dermal tissue in step 110 as the swollen, turgid dermal tissue eases passage of an inner tubular member without scar-inducing cutting or tearing of the dermis.

Preoperative treatment 100 of the donor area can also include spreading the dermal tissue surrounding individual hairs with the use a of a donor site preparation instrument as discussed above with respect to FIGS. 2C and 3A. The preparation instrument could be advanced around one or more donor hairs to prepare dermal tissues surrounding those hairs to receive an inner tubular member. The preparation instrument could be configured with a polished end sized and configured to dilate a pore surrounding a hair to allow passage of an inner tubular member in the pore.

In step 110, an inner tubular member or guide can be slidably applied to a hair in the donor area. The inner tubular member can be advanced down the shaft of the hair and through the dermal layer, its advancement through the dermis eased by the tissue turgidity and spreading of the dermal tissue as noted above.

Using a sterile technique, an operator can grasp the guide and slide a blunt distal end of the guide over an end of a hair in the donor area. Once the hair exits the opposite end of the guiding inner tubular member, the operator can grasp the hair shaft, possibly using small forceps, in one hand and will advance the guide down the hair shaft using the other hand.

Because of the tissue turgidity, the guide is able to be introduced deep within the dermal tissues. Preferably, the inner tubular member is configured such that its inner diameter is larger than a hair shaft diameter but smaller than a diameter of the hair follicle bulb. Thus, in step 110, when the guiding inner tubular member has been advanced to an upper surface of the hair follicle bulb, the inner tubular member will no longer be capable of being advanced further.

The inner tubular member, preferably has a blunt distal end so that the distal end of the inner tubular member does not injure the hair follicle bulb. Thus, the guiding inner tubular member will follow the hair shaft and seek the hair follicle bulb. When the guide reaches an upper surface of the hair follicle bulb, a palpable resistance is felt, and the inner tubular member has been fully advanced.

FIG. 3B illustrates an inner tubular member according to certain embodiments of hair transplantation device having been fully advanced into dermal tissues according to step 110. While reference is made to the hair transplantation device according to embodiments described herein, it is contemplated that the hair removal method could be implemented with other hair transplantation devices.

In step 120, an outer tubular member can be slid over the hair in the inner tubular member and slid until the distal end of the outer tubular member is aligned with the distal end of the inner tubular member at an outer surface of the hair follicle bulb. The operator can grasp the guide in one hand and the outer tubular member in the other hand and advance the outer tubular member over the guide. A visual indicator on the guiding inner tubular member can indicate when the outer tubular member has been advanced such that its distal end is aligned with the distal end of the inner tubular member. Alternatively, the advancement of the outer tubular member such that its distal end is aligned with the distal end of the inner tubular member can trigger an audible or tactile cue can to be generated by mating features on the inner tubular member and outer tubular member.

In step 120, as the outer tubular member is slid into the dermal tissue surrounding the hair, an inner dilating portion of the outer tubular member dilates the dermal tissue, thus reducing the risk of scarring in the hair removal procedure. Once the outer tubular member is slid into the above-described distal end alignment with the inner tubular member, an outer harvesting portion of the outer tubular member is advanced to dilate the pore surrounding the hair follicle bulb and cut the dermal tissue surrounding the hair follicle bulb.

A stop or other feature can be provided on the outer tubular member to limit the advancement travel of the outer harvesting portion relative to the inner dilating portion so that the outer harvesting portion cuts substantially only the dermal tissue surrounding the hair follicle bulb. Once the hair follicle bulb has been separated from the dermal tissues, the inner tubular member and the dilating portion of the outer tubular member can be withdrawn from the donor area, leaving only the harvesting portion of the outer tubular member. FIGS. 4A, 4C and 5A depict outer tubular members 30 according to various embodiments of hair transplantation device being advanced over a hair 22 to separate the follicle from the dermal tissue.

The outer tubular member can then be withdrawn with the hair follicle in step 130. In certain embodiments, a suction device can be used to assist withdrawal of the hair follicle with the outer tubular member. The suction device can include a generally cylindrical housing with a depressible elastic domed surface as described with reference to FIG. 6.

To use the exemplary suction device 50, the operator can depress the elastic domed surface 56, then, while maintaining the elastic dome in a depressed state, applies the suction device to a sealing surface such as an enlarged collar on the outer tubular member or a mount plate providing an interface with the outer tubular member. Once a sealing contact has been formed between the suction device and the outer tubular member, the operator releases the elastic dome to create suction in the harvesting portion of the outer tubular member. This suction pulls and holds the hair follicle bulb in the outer tubular member. The operator can then remove the outer tubular member from the donor area using a gentle rotational motion on the outer tubular member.

In step 140, post-operative treatment can be provided to the donor area. The post-operative treatment promotes healing of the dermal tissue that has been cut and reduces the risk of infection. Post-operative treatment can include administering a topical ointment or applying a gauze pad or another light dressing. A bandage can be provided especially if the method of the present invention is repeated for multiple adjacent hairs in the donor area. It should be noted that post-operative care of the donor area is minimal since there are essentially no open wounds (the pores that have been dilated during harvest will return to normal size relatively quickly after removal of the hair follicle). Once the donor area has healed from the hair follicle removal procedure, substantially no post-operative scarring will occur.

Hair Implantation

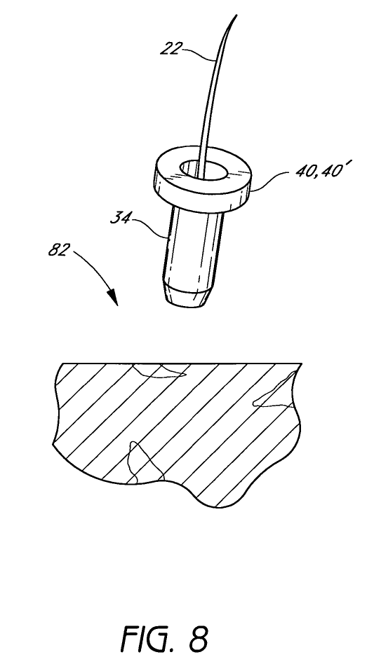

FIG. 8 depicts a recipient area 82 being a bald patch of skin with the harvester 34, positioned for transplant of a hair 22. Although depicted as having a collar 40 having a generally disk shape, a harvester 34 having a modified collar 40′ with a trapezoidal cross-section, a harvester 34 having a collar of a profile, or a harvester 34 without a collar can be used to implant a hair 22 in a recipient area. Advantageously, if the same harvester 34 is used to both remove and implant a hair follicle, risk of damage to the hair follicle during its removal from the outer tubular member 30 or relocation to the recipient area 82 is greatly reduced over hair transplantation methods in which skin from a donor area must be segmented into smaller units before it can be implanted in a recipient area.

FIG. 9 depicts a close-up view of the hair recipient area 82 after an incision 90 has been made in the hair recipient area 82 in preparation for implantation of the hair follicle. The incision 90 can be with a small scalpel tip or tangentially sharpened needle. It is contemplated that techniques known in the art for implanting a hair follicle in a recipient area can be used with various embodiments of hair transplantation device described herein. Although an incision-based technique is depicted herein, the use of a hair transplantation device to implant a hair is not so limited.

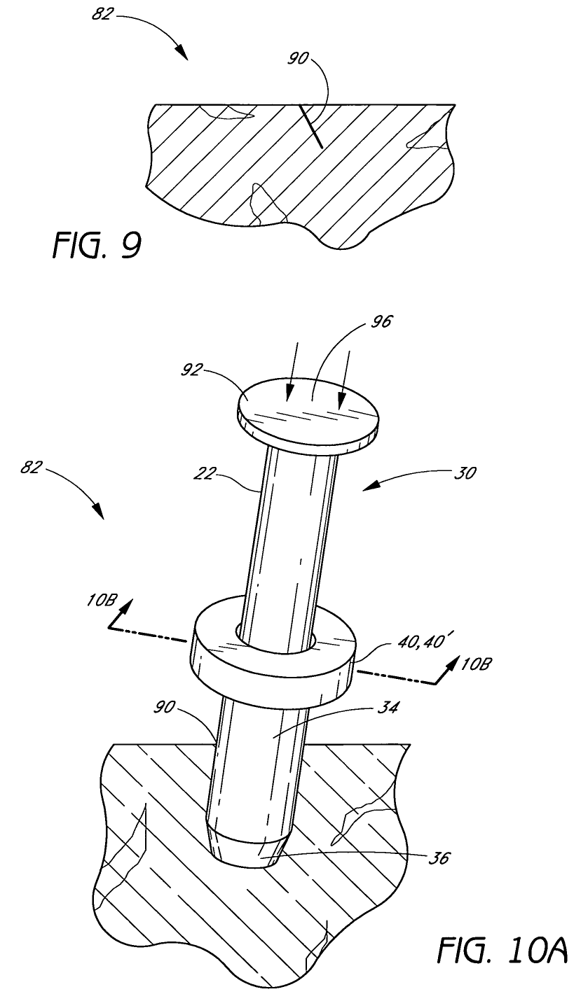

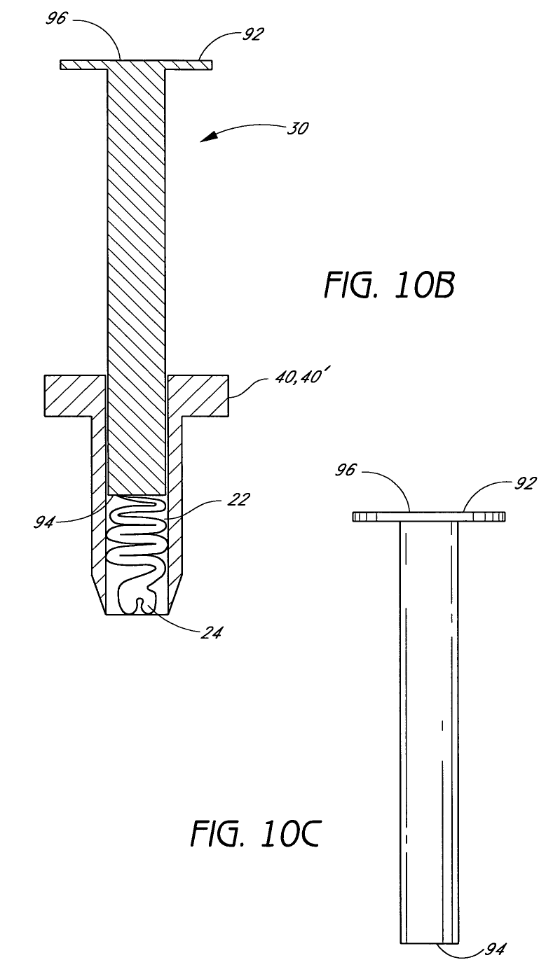

FIG. 10A depicts a harvester 34 of an embodiment of hair transplantation device invention advanced into the incision 90 in the recipient area 82. As described in more detail above regarding the removal of a hair follicle, the distal end 36 of the harvester 34 spreads the dermal tissues surrounding the incision site 90 as it is advanced. Also depicted in FIG. 10A is a plunger device 92 configured for use in hair transplantation devices. The plunger device 92 is slidably advanceable through the harvester 34. A cross-sectional side view of the plunger device 92 as advanced through the harvester 34 depicted in FIG. 10B. FIG. 10C depicts a side view of the plunger device 92.

The plunger device 92 can comprise a generally cylindrical body having a blunt distal end 94. The plunger device can also feature a substantially flat surface 96 configured to allow the user to apply an axial force to the plunger. Preferably, the plunger device 92 has an diameter configured to closely match the inner diameter of the harvester 34. Thus, advancing the plunger 92 into the harvester 34 extrudes a hair 22 from the harvester 34.

The blunt distal end 94 of the plunger device 92 reduces a risk of injury to the hair follicle bulb 24 during the extrusion process. Once a hair 22 and its follicle bulb 24 have been extruded from the outer tubular member 30 into recipient area 82, the harvester 34 can be withdrawn from the skin at the hair recipient area 82. Although a substantially cylindrical plunger device is depicted herein, it should be recognized that alternate configurations of plunger devices could be used with the hair transplant device.

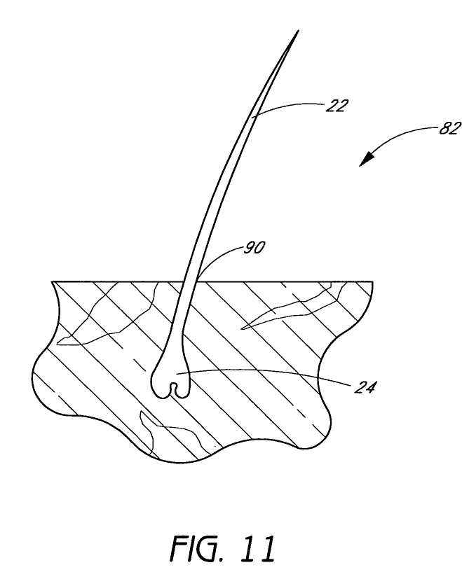

FIG. 11 depicts the hair 22 as implanted in an incision 90 in the hair recipient area 82 after the harvester 34 has been withdrawn. The transplanted hair follicle 24 can now heal into the dermal tissue of the recipient area 82.

Hair Transplantation Method

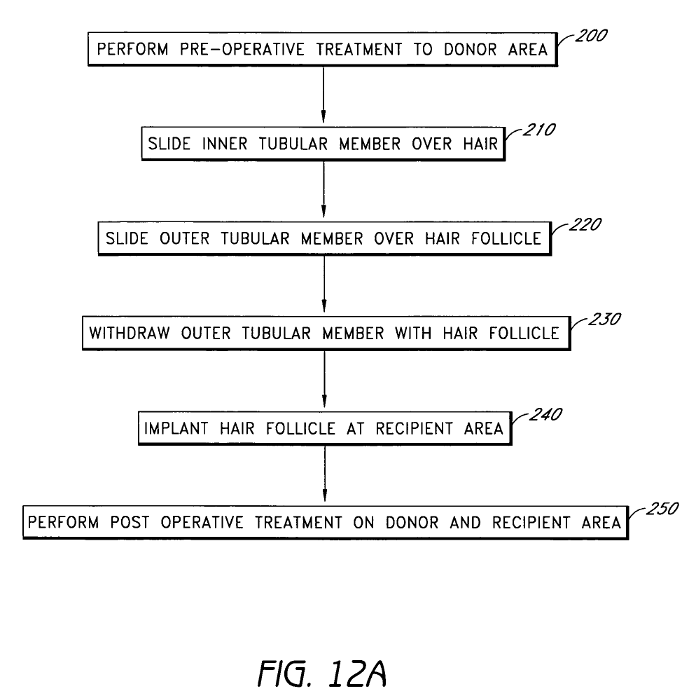

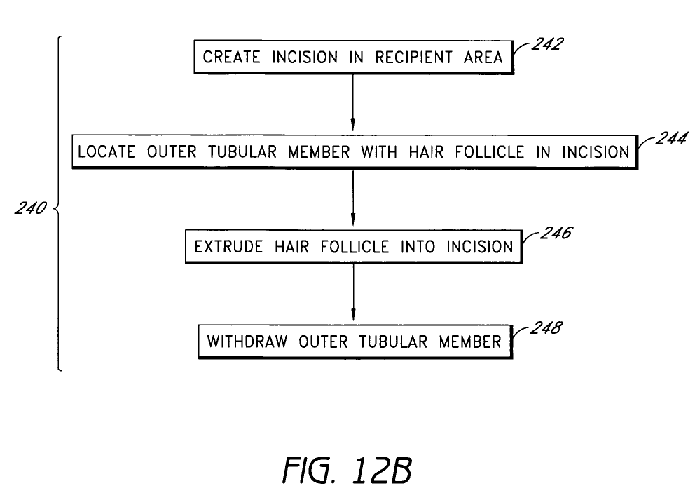

Other embodiments of the inventions disclosed herein relate to hair transplantation methods that can be implemented using the hair transplantation device disclosed herein, or other hair transplantation devices. FIG. 12A illustrates steps comprising certain embodiments of air transplant method of the present invention. Steps 200-230 of the depicted hair transplantation method include performing steps of the hair removal method discussed with respect to FIG. 7.

In step 200, pre-operative treatment is provided to a hair donor area. The pre-operative treatment can include administering an anesthetic to the hair donor area to render the donor area tissue turgid. This tissue turgidity increases the ability of the hair transplant device to remove a hair follicle without scarring the donor area tissue. The pre-operative treatment can also include preparing the donor site with a donor site preparation instrument such as is described with respect to FIGS. 2C and 3A. In step 210, a guiding inner tubular member is slidably advanced over a donor hair. As described above regarding hair removal methods, the guide is advanced until it reaches an upper surface of the hair follicle bulb of the donor hair. The inner tubular member is configured to have an inner diameter such that it cannot be advanced over the hair follicle bulb. The inner tubular member can also be configured with a blunt distal end such that distal end of the inner tubular member will not substantially harm the hair follicle bulb.

In step 220, an outer tubular member is slid over the inner tubular member over the hair and advanced to a depth in the dermal tissue of the donor area such that the distal end of the outer tubular member is aligned with the distal end of the inner tubular member. The hair transplantation device can be configured such that the alignment of distal ends of the inner and outer tubular members generates a visual cue, a tactile cue, or an audible cue, thus notifying the user that the proper alignment has been attained.

As discussed above, with reference to the hair removal method, the outer tubular member can have an inner dilating portion that spreads the dermal tissue around the donor hair. One advantage of this inner dilating portion is that it reduces the risk of scarring to the donor area by dilating the dermal tissue around the hair shaft rather than cutting the tissue. The outer tubular member can have an outer harvesting portion configured to be slidably advanced over the inner dilating portion to dilate the pore surrounding the hair follicle bulb and cut the dermal tissues surrounding the hair follicle bulb. Since the outer tubular member has been slidably applied over the inner tubular member down to a depth of the upper surface of the hair follicle bulb, the outer harvesting portion of the outer tubular member is properly centered and aligned with the hair follicle bulb. One advantage of this proper centering and alignment is that the outer harvesting portion will cut the dermal tissue around the hair follicle bulb rather than the hair follicle bulb itself. Once the dermal tissue around the hair follicle bulb has been cut, in step 230, the outer tubular member is withdrawn with the hair follicle.

A suction device such as is described above in the discussion of the hair removal device can be used to assist this withdrawal. The outer tubular member can then be relocated to a recipient area on the patient. FIGS. 4A, 4C, and 5A depict outer tubular members 30 according to various embodiments of hair transplantation device being advanced over a hair 22 to separate the follicle from the dermal tissue.

In step 240, the hair follicle is implanted at the recipient area. In the hair transplantation method, the harvesting portion of the outer tubular member can be used to implant the hair follicle into the recipient area or other devices for hair implantation can be used. Advantageously, by using the outer tubular member as a hair removal, transport, and implantation tool, the risk of injuring the hair follicle during is minimized as the hair follicle is enclosed in the outer tubular member throughout much of the process.

Step 240, implanting a hair follicle at the recipient area can further comprise additional steps. FIG. 12B is a flow chart depicting the steps further comprising a hair implantation step 240 using the outer tubular member to implant the hair follicle. In step 242, an incision is created in the recipient area. The incision can be created with a small scalpel tip or a standard tangentially sharpened needle. It should be noted that although the hair implantation step 240 employs an incision for implantation, other methods can be used in conjunction with an outer tubular member to implant the hair follicle in a recipient area. Once the incision has been created in step 242, the distal end of the harvester of the outer tubular member containing the hair follicle is advanced into the incision in step 244. In step 246, the hair follicle is extruded from the outer tubular member into the incision. A plunger device can be used to extrude the hair follicle into the incision. Once the hair follicle has been extruded into the incision, the outer tubular member is withdrawn in step 248. FIGS. 8-11 illustrate the implementation of steps 242, 244, 246, and 248 using an embodiment of hair transplantation device described above with respect to FIGS. 1-6.

Once the hair follicle has been implanted at the recipient area in step 240, post-operative treatment is to be provided to the donor area and recipient area in step 250. The hair transplantation method of the present invention can be repeated multiple times until a desired number of hairs have been added to the recipient area. However, a hair transplantation device is preferably discarded after use on a single hair. The hair transplantation device is no longer be sterile following use: surfaces of the inner tubular member and the outer tubular member can be coated with blood or dermal tissue scrapings. Additionally, reuse of the outer tubular member can lead to ripping of dermal tissue as the honed tip of the harvester can lose some of its initial sharpness through use. If the donor area has a relatively high hair concentration, the steps of performing pre-operative treatment 200 and perform post operative treatment 250 can only need to be performed a single time, while steps 210 through 240 can be performed one time for each hair follicle transplanted from the donor area to the recipient area. As noted above, the donor area can require only minimal post-operative care since there are essentially no open wounds. The recipient area requires post-operative care generally appropriate for a dermal incision including application of topical ointments and dressings.

Although these inventions have been disclosed in the context of certain preferred embodiments and examples, it will be understood by those skilled in the art that the inventions extend beyond the specifically disclosed embodiments to other alternative embodiments and/or uses of the invention and obvious modifications and equivalents thereof. Further, the various features of these inventions can be used alone, or in combination with other features of the inventions other than as expressly described above. Thus, it is intended that the scope of the inventions herein disclosed should not be limited by the particular disclosed embodiments described above, but should be determined only by a fair reading of the claims that follow.

What is claimed is:

1. A method for removing a hair follicle from an animal comprising:
sliding a first tubular member over a single hair of the animal until the first tubular member reaches an upper end of a follicle of the hair;
sliding a second tubular member over the first tubular member and into a dermis of the animal surrounding the hair follicle to a position deeper into the dermis than the upper end of the follicle; and
withdrawing the second tubular member with the hair follicle from the dermis.

2. The method of claim 1, further comprising the step of injecting an anesthetic solution into a surface of skin of the animal adjacent a hair follicle to be removed before the step of sliding a first tubular member over a single hair of the animal.

3. The method of claim 1, wherein the step of injecting comprises injecting an amount of anesthetic sufficient to palpably stiffen the dermis.

4. The method of claim 1, further comprising the step of providing post-operative treatment to an area adjacent the withdrawn hair follicle after the step of withdrawing the second tubular member with the hair follicle.

5. The method of claim 1, wherein the step of withdrawing the second member with the hair follicle further comprises the step of creating a vacuum in the second tubular member with a suction device sealably mated with the second tubular member.

6. A method for transplanting a hair follicle from an animal comprising:
sliding a first tubular member over a single hair of the animal located at a first location on the animal until the first tubular member reaches an upper end of a follicle of the hair;
sliding a second outer tubular member over the first tubular member and into the dermis of the animal surrounding the hair follicle to a position beyond the upper end of the follicle;
withdrawing the second tubular member with the hair follicle from the dermis;
implanting the hair follicle into a second location on the animal.

7. The method of claim 6, wherein the step of implanting the hair follicle further comprises the steps of:
creating an incision in the second location on the animal;
inserting the second tubular member into the incision at the second location;
discharging the hair follicle from the second tubular member.

8. The method of claim 7, wherein the step of discharging the hair follicle from the second tubular member comprises the step of inserting a plunger into an inner diameter of the second tubular member.

9. The method of claim 6, further comprising the step of injecting an anesthetic solution into a surface of skin of the animal adjacent a hair follicle to be removed before the step of sliding a first inner tubular member over a single hair of the animal.

10. The method of claim 6, wherein the step of injecting comprises injecting a sufficient quantity of anesthetic to palpably stiffen the skin.

11. The method of claim 6, further comprising the step of providing post-operative treatment to the first location and the second location.

12. A medical device comprising:
A first tubular member having a blunt distal end; and
A second tubular member having a distal end configured to spread dermal tissue of an animal and cut dermal tissue around a hair follicle of the animal and wherein the second tubular member is configured to be slidably applied around the first tubular member.

13. The device of claim 12, wherein the second tubular member further comprises:
a dilating portion having a distal end configured to dilate dermal tissue of an animal and wherein the dilating portion is configured to be slidably applied around an outer surface of the first tubular member; and
a harvesting portion having a distal end configured to cut dermal tissue of an animal around a hair follicle of the animal and wherein the harvesting portion is configured to be slidably engaged with the dilating portion of the second tubular member.

14. The device of claim 12, wherein the first tubular member has an inner diameter configured such that the first tubular member is slidable around a single hair of an animal and the first tubular member is not slidable around a follicle of the single hair.

15. The device of claim 14, wherein the first tubular member has an outer diameter configured such that the first tubular member removes substantially no dermal tissue of the animal when the first tubular member is slid over the shaft of the single hair.

16. The device of claim 12, wherein the first tubular member has an inner diameter of less than approximately 0.5 mm in diameter.

17. The device of claim 12, wherein the first tubular member comprises a 27 gauge flexible tube.

18. The device of claim 13, wherein the dilating portion of the second tubular member comprises a 22 gauge tube.

19. The device of claim 12, wherein the distal end of the second tubular member has a diameter of approximately 1 mm.

20. The device of claim 12, wherein the harvesting portion of the second tubular member comprises an 18 gauge tubular member.

21. The device of claim 12, wherein the second tubular member comprises a collar at a proximal end of the second tubular member.

22. The device of claim 21, wherein the collar comprises a sealing surface configured to sealably mate with a suction device.

23. The device of claim 22, further comprising a removable suction device configured to mate with the mating surface of the collar.

24. The device of claim 23, wherein the removable suction device comprises a housing;
a sealing edge disposed on the housing and configured to sealably mate with the sealing surface of the collar; and
a deformable wall mounted on the housing.

25. The device of claim 24, further comprising a mounting plate having a sealing surface configured to sealingly couple with the sealing edge of the suction device and an aperture disposed in the sealing surface and configured to sealingly couple to the collar.

26. The device of claim 12 further comprising a plunger configured to fit within an inner diameter of the second tubular member and configured to discharge a hair follicle from the distal end of the second tubular member.

27. A medical device comprising:
a guide configured to locate a hair follicle bulb in dermal tissue of an animal; and
a first tubular member configured to be slidably engaged around the guide;
wherein the first tubular member comprises a dilator and a harvester, and wherein the harvester is slidably advanceable with respect to the guide such that the harvester can cut the dermal tissue surrounding the hair follicle bulb.

28. The medical device of claim 27, wherein the guide has an inner diameter sized and configured to allow the guide to be slidably advanced over a hair shaft but not a hair follicle.

29. The medical device of claim 27, wherein the dilator has a distal end configured to spread dermal tissue surrounding a hair shaft, and wherein the harvester has a distal end configured to cut dermal tissue surrounding a hair follicle bulb.
